(19) United States
(12) Patent Application Publication
(10) Pub. No.: US 2026/0241135 A1
(43) Pub. Date: Aug. 20, 2026

(54) A haptic device

(71) Applicant: Aalto University Foundation sr, Aalto (FI)

(72) Inventors: Jukka Planman, Aalto (FI); Juliana Harkki, Aalto (FI); Pauli Tuovinen, Aalto (FI); Heikki Nieminen, Aalto (FI)

(21) Appl. No.: 18/874,595

(22) PCT Filed: Jun. 13, 2023
(86) PCT No.: PCT/FI2023/050351

§ 371 Date: Dec. 13, 2024

(30) Foreign Application Priority Data
Jun. 13, 2022 (FI) 20227088

(51) Int. Cl. A61M 21/00; B06B 1/06

(52) U.S. Cl. CPC A61M 21/00; B06B 1/0622; A61M 2021/0022; A61M 2205/0294; B06B 2201/76

(57) ABSTRACT

There is provided a haptic device. The haptic device comprises an electric control unit and one or more transducer element. The one or more transducer element is configured to be activated based on electrical signaling from the electronic control unit. Configuration of the one or more transducer element is such that that vibrations are obtained through modifying electrical signaling from the electronic control unit to the one or more transducer element such that if the device is arranged against a skin of a user, the vibrations are configured to generate pressure fields towards the skin of the user.

### TECHNICAL FIELD

The present invention relates to a haptic device configured to provide a haptic feedback to a user of the device.

### BACKGROUND

Chronic alcohol use leads to long-term changes in the brain circuits that drive reward, motivation, and stress. These alterations in neural functioning result in behavioral changes and a high risk of relapse even after long abstinence. This makes alcohol use disorder, AUD, an extremely challenging disease to treat. In fact, up to 85% of those who attempt to quit drinking with current pharmacological and psychosocial treatments will eventually relapse.

Alcohol craving is one of the behavioral results of alcohol-induced neural adaptations. It is a characteristic symptom of AUD and a major reason for relapses. Alcohol craving may be triggered by emotional stress, anxiety, and arousal.

Psychosocial therapies and cognitive behavioral therapies, CBT, are the cornerstones of the modern treatments. However, the waiting times for the treatments may be unacceptably long. This may lower the motivation to quit drinking and lead to a relapse. In addition, psychosocial therapies involve a high risk of experienced stigma and high specialized care costs.

Pharmacological therapies have been shown to reduce harmful drinking behavior and act as a supporting factor with psychosocial therapies to maintain abstinence. However, their efficacy is modest and dependent on the correct timing in relation to alcohol consumption. Furthermore, there is a possibility of side effects, and the cost of medication can become high in the long term without reimbursement.

Current AUD treatments rely on psychosocial, cognitive behavioral, CBT, and pharmaceutical therapies either as standalone or combination treatments. However, the exceptionally high relapse rate of 85% suggests that state-of-the-art treatments are highly ineffective at preventing relapse. Moreover, the cost of treatments can become too high for AUD customers to afford without reimbursement and significantly obstruct access to treatments. Lowering the experienced stigma and offering quick access to the treatment at low cost are some of the most important improvements needed for the current standard of care.

### SUMMARY

Aim is to provide a haptic device for a self-care.

In addition to the gap for device therapy solutions in healthcare system, currently, there are no self-care therapeutic solutions available for reducing harmful drinking behavior.

The invention is defined by the features of the independent claims. Some embodiments are defined in the dependent claims.

According to a first aspect of the present invention, there is provided a haptic device, comprising an electric control unit and one or more transducer element, wherein the one or more transducer element is configured to be activated based on electrical signaling from the electronic control unit, and wherein the one or more transducer element is configured such that that vibrations are obtainable through modifying electrical signaling from the electronic control unit to the one or more transducer element such that the vibrations are configured to generate pressure fields.

Various embodiments may include one or more of the following:

According to an embodiment, the one or more transducer element is configured such that one or more eigenfrequencies are obtainable, and shifts between the eigenfrequencies are enabled with the same configuration through modifying electrical signaling from the electronic control unit to the one or more transducer elements, wherein the shifts between eigenfrequencies cause moving of locations of maximum pressure fields.

In an embodiment, one or more transducer element is configured to operate in a unimodal, a bimodal, or a trimodal vibration mode. In addition or alternatively, the one or more transducer element is configured to operate either in transverse or in longitudinal mode. The one or more transducer element may comprises a piezo element.

The device may further comprise at least one membrane. The one or more transducer element may be coupled to the at least one membrane. The at least one membrane may be configured to be vibrated with the one or more transducer element in order to obtain plural different eigenfrequency patterns in the at least one membrane. The one or more transducer element is coupled on top of the at least one membrane, and/or at sides of the at least one membrane. A resonance pattern in the at least one membrane may be configured to generate spatiotemporally modifiable local displacement regions, which generate acoustic pressure fields respectively. The at least one membrane may be configured to thin in its vertical or top-bottom direction; and/or in its horizontal or side direction.

In an embodiment, at eigenfrequency antinodes, vibration of the at least one membrane is configured to vibrate such that vibration of the at least one membrane exhibit sufficient boundary displacements to generate pressure fields.

According to an embodiment the device comprises at least one adjacent membrane layered together with the at least one membrane. The at least two membranes may be layered together and coupled to only one transducer. The layered membranes may be configured to oscillate longitudinally, and the layered membranes have different acoustic impedances.

In an embodiment, the device comprises one or more fibers coupled to the one or more transducer element. The electronic control unit is configured to supply appropriate electrical signal to the one or more transducer element in order to control transmitted flexural mode in the one or more fibers. The one or more fibers may have uniform diameter along the length of the fibers, or the diameter may decrease towards the point of the fibers.

The device may comprise at least one impedance matching element, wherein the one or more fiber is directly coupled with the one or more transducer element with the impedance matching elements.

The device may comprise a solenoid.

In an embodiment, the device comprises at least two or more casings comprising one or more transducer elements; with or without one the following: the at least one membrane, or the one or more fibers. The at least two or more casings may be sequenced in order to control time difference between activations.

The device may be a wearable device, or the device is comprised in a furniture or the device a in a brush-like device, wherein vibrated elements arranged against a skin of the user are configured to provide haptic forces.

The device may comprise extrusion elements configured to maintain a constant distance between a surface of a skin of a user and the casing. The at least one membrane may be placed in less than 2 mm distance from a surface of a skin of a user. The extrusion element may be free to oscillate.

The one or more transducer element may be configured to oscillate longitudinally and arranged to actuate the one or more fibers in a transverse mode such that the extrusion element is used as a vibrating membrane or as a waveguide.

In an embodiment, acoustic resonances employed in the at least one membrane are configured to generate pressure fields at air-skin interface, wherein difference in acoustic impedance is configured to result in acoustic radiation force acting towards user skin. Alternatively, acoustic resonances employed in the at least one membrane are configured to generate pressure fields at a surface of a skin of a user, which is configured to transfer acoustic energy through coupling between the one or more fibers and user skin hair.

The one or more transducer element may be configured to generate transverse displacement which generates flexural waves in the one or more fibers, wherein the flexural waves of the one or more fibers are coupled to skin hairs of the user, which is configured to cause tissue deformation at the hair root and follicle.

The device may comprise plurality of casings in a cellular structure, wherein the activation of the plurality of casings may be sequenced in order to provide continuous haptic patterns.

The device may comprise one or more sensors configured to provide electrical signaling to the electronic control unit, wherein the one or more sensors may be configured to measure a distance—either from the one or more transducer, or from the at least one membrane—to a skin surface of the user, and—biomarkers obtained from the one or more sensors. The biomarkers comprise at least one of the following: blood pressure, heart rate, heart rate variability, HRV and skin electrodermal activity. Activity of the obtained biomarkers is used in biofeedback to monitor activation of the device. The two or more transducer elements may be activated individually in synchronous or asynchronous patterns based on the electrical signaling from the electronic control unit and/or based on the biofeedback. Data from the device and the one or more sensors is exportable to a mobile application and/or to a desktop application.

The device comprises a haptic device, which may be used for therapeutic purposes.

### BRIEF DESCRIPTION OF THE DRAWINGS

In the following embodiments are discussed in more detail with reference to the attached drawings, of which:

FIG. 1 illustrates, by way of an example, an arrangement for a device according to an embodiment.

FIG. 2 illustrates, by way of an example, an arrangement for a device according to an embodiment.

FIG. 3 illustrates, by way of an example, an arrangement for a device according to an embodiment.

FIG. 4 illustrates, by way of an example, an arrangement for a device according to an embodiment.

FIG. 5 illustrates, by way of an example, an arrangement for a device according to an embodiment.

FIG. 6 illustrates, by way of an example, an arrangement for a device according to an embodiment.

FIG. 7 illustrates, by way of an example, an arrangement for a device according to an embodiment.

FIG. 8 illustrates, by way of an example, an arrangement for a device according to an embodiment.

FIG. 9 illustrates, by way of an example, an arrangement for a device according to an embodiment.

FIG. 10 illustrates, by way of an example, an arrangement for a device according to an embodiment.

FIG. 11 illustrates, by way of an example, an arrangement for a device according to an embodiment.

FIG. 12 illustrates, by way of an example, an arrangement for a device according to an embodiment.

FIG. 13 illustrates, by way of an example, an arrangement for a device according to an embodiment.

FIG. 14 illustrates, by way of an example, an arrangement for a device according to an embodiment.

FIG. 15 illustrates, by way of an example, an arrangement for a device according to an embodiment.

FIG. 16 illustrates, by way of an example, an arrangement for a device according to an embodiment.

FIGS. 17A, 17B, 17C and 17C illustrate, by way of an example, a device according to an embodiment.

Figures are presented as illustrative examples and embodiments may not be limited solely to the illustrated parts, but modifications may be made under the scope as defined in the claims. Figures that may not fully present the claimed invention, aim to provide better understanding on the context and relating technical field.

### DESCRIPTION OF EMBODIMENTS

The solution is a ‘neurodesigned’ therapeutic haptic device that produces tactile stimulation on the surface of the skin for neuromodulation. By optimizing the response to stimulus parameters of the active component of the device, such as but not limited to speed, force, temperature, acceleration, activation interval, and direction of motion, the device achieves a positive physiological and behavioral response in a subject in a very short time. We target at combining and modulating these parameters to adapt to interpersonal differences.

In the following, an apparatus and actuation mechanisms utilizing acoustic energy are described: 1) Employment of acoustic resonances in a vibrating element, 102; 104; 105; 106; 107; 110; 112, to generate pressure fields at skin's surface to evoke C-tactile afferents, CT, neural firing, and 2) transfer of acoustic energy through coupling between a fiber, 115, or monofilament, 115, (hereon referred as fibers) and hair to evoke neural firing in CTs.

Throughout this application a vibrating element may be a transducer, a membrane, a vibration membrane, a thin membrane, or a hair. Hair refers to skin hair, for example a vellus hair or a terminal hair. A device refers to a haptic device, which may be used for therapeutic purposes. An apparatus may be used as a synonym to a device. Coupling and connecting may be used as synonyms. A transducer may be vibrated via external force. An arrangement, a casing or an active element for a (haptic) device comprises one or more transducer. Additionally, the arrangement may comprise at least one membrane or fiber(s).

FIG. 1 illustrates, by way of an example, an arrangement for a device according to an embodiment. The device comprises transducer elements 100 coupled to a membrane 102. The transducer elements 100 are coupled on top of the membrane 102. The transducer elements 100 are activated using electric signalling. The activated transducer elements 100 cause vibration of the membrane 102. A vibration pattern of the membrane 102 is illustrated as a dashed line 103.

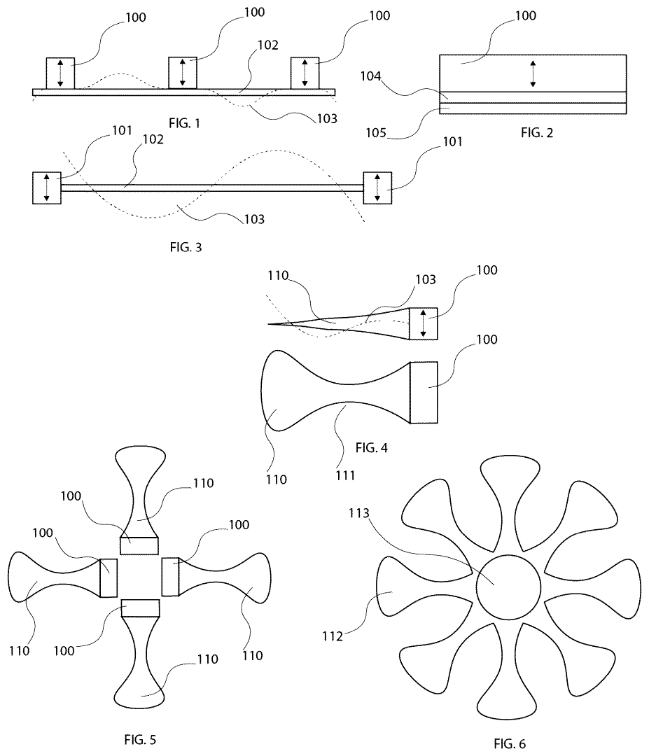

FIG. 2 illustrates, by way of an example, an arrangement for a device according to an embodiment. The device comprises a transducer element 100 coupled to layered membranes 104, 105. The transducer element 100 is longitudinally oscillating, as illustrated by an arrow in FIG. 2. Acoustic impedances of the layered membranes 104, 105 is smaller compared to an acoustic impedance of the transducer element 100. An acoustic impedance of the outer layer 105 is smaller than an acoustic impedance of the middle layer 104, which in turn is smaller than an acoustic impedance of the transducer element 100. This may enable to amplify boundary displacement generating pressure fields.

Longitudinally oscillating transducer element 100 can be layered with materials having smaller acoustic impedances 104, 105 compared to 100 to amplify boundary displacement generating pressure fields. Acoustic impedances of the layers 100, 104, 105 must be in scale of Z1>Z2>Z3, where Z1 is for the transducer element 100, Z2 for a layer 104, and Z3 for the layer 105. The layers 104, 105 must have thickness less than the wavelength of the longitudinal wave.

FIG. 3 illustrates, by way of an example, an arrangement for a device according to an embodiment. The device comprises transducer elements 100 coupled at sides of a membrane 102. The transducer elements 100 are activated using electric signalling. The activated transducer elements 100 cause vibration of the membrane 102. A vibration pattern of the membrane 102 is illustrated as a dashed line 103.

The haptic device, or an apparatus, may be used for therapeutic methods. The following apparatus and a method for C-tactile afferents, CT, activation comprises of one or more transducer elements 100, 101, 113 coupled to a thin membrane 102, 104, 105, 106, 107, 110, 112 with impedance matching elements 107, 108, to optimize energy transmission into a vibrating geometry. The transducer elements 100, 101, 113 may operate either in transverse 101 or longitudinal 100 mode in unimodal (one-directional), bimodal (two-directional), or trimodal (three-directional) vibration mode. The transducer elements may be coupled on top (FIG. 1) or at the sides (FIG. 3) of the membrane in various configurations to obtain several different eigenfrequency patterns in the membrane 102, 103, 104, 105, 106, 107, 110, 112. A plausible vibration pattern for a geometry is described by 103. The configuration of transducer elements 100, 101, 113 is selected so that one or more eigenfrequencies can be obtained and instantaneous shift between them is possible with the same configuration through modifying electrical signaling from electronic control unit to the transducer elements 100, 101, 113.

FIG. 4 illustrates, by way of an example, arrangements for a device according to an embodiment. The upper arrangement of FIG. 4 comprises a transducer element 100 and a membrane 110. Movement of the transducer element 100 is illustrated by an arrow, and the corresponding vibration caused to the membrane 110 is illustrated by a dashed line 103. The membrane 110 has an elongated form, thinning towards its free end, which is opposing the end at which the membrane 110 is coupled to the transducer 100. The lower arrangement of FIG. 4 comprises a transducer element 100 and a membrane 110. The membrane 110 has an elongated form, thinning at the middle 111 of the membrane 110. The both membranes 110 of FIG. 4 have been designed thinning in vertical direction. The thinning is designed towards a location where resonance amplitude maximum is desired. This enables amplifying membrane displacement by acoustic energy.

FIG. 5 illustrates, by way of an example, an arrangement for a device according to embodiments. The arrangement comprises number of transducer elements 100 coupled to membranes 110, respectively. The membranes 110 are thinning in their horizontal direction, or at the middle 111 of an elongated membrane. This arrangement may enable creating predefined flexural wave nodes, which in turn may cause amplifying the membranes 110 displacement at free ends (distal ends) of the membranes 110.

FIG. 6 illustrates, by way of an example, an arrangement for a device according to embodiments. The arrangement comprises a transducer element 113 and membranes 110. The membranes 110 have been merged. This enables providing different geometries and/or complex shapes for the merged membranes 110. The merged membranes 110 may be coupled to a less number of transducer elements 113 than the number of the merged membranes 110. The merged membranes may be coupled to a single transducer element 113.

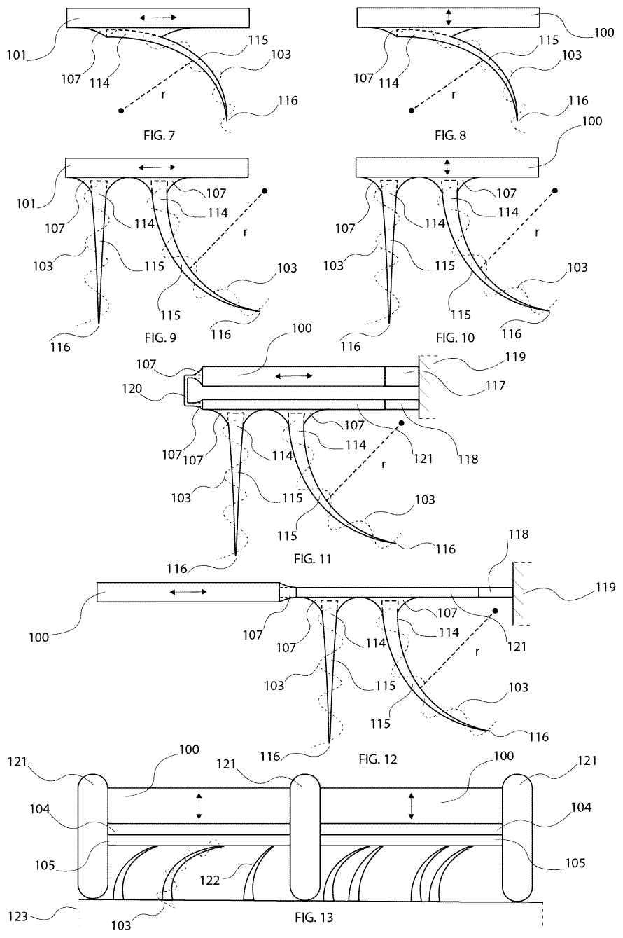

FIG. 7 illustrates, by way of an example, an arrangement for a device according to an embodiment. The arrangement comprises a transducer element 101 and a fiber 115 connected to the transducer element 101 via an impedance matching element 107. Fiber 115 is connected to the transducer element so that a proximal fiber 114 is aligned parallel to a surface of the transducer element 101, and narrowing towards its opposing end 116, which is configured to contact with skin hairs. A vibration pattern of the membrane 102 is illustrated as a dashed line 103. The transducer 101 is configured to oscillate transversely as illustrated by an arrow in FIG. 7.

FIG. 8 illustrates, by way of an example, an arrangement for a device according to an embodiment. The arrangement comprises a transducer element 101 and a fiber 115 coupled to the transducer element 101 via an impedance matching element 107. Fiber 115 is connected to the transducer element so that a proximal fiber 114 is aligned parallel to a surface of the transducer element 101, and narrowing towards its opposing end 116, which is configured to contact with skin hairs. A vibration pattern of the membrane 102 is illustrated as a dashed line 103. The transducer 100 is configured to oscillate longitudinally as illustrated by an arrow in FIG. 8.

Vibrations or resonance may be caused by an external force or without such. Eigenfrequency is also known as natural frequency. Eigenfrequency is the frequency at which a system tends to oscillate in the absence of any driving force. The motion pattern of a system oscillating at its eigenfrequency is called a normal mode, where all parts of the system move sinusoidally with the same frequency. If the oscillating system is driven by an external force at the frequency at which the amplitude of its motion is greatest (close to natural frequency of the system), this frequency is called resonant frequency.

Different eigenfrequencies may also be achieved by modifying the shape of the membrane 102, 104, 105, 106, 107, 110, 112. Different shapes may include primitives such as circles and squares but also complex shapes. In order to amplify a membrane 106, 107, 110, 112 displacement by acoustic energy, the membrane 106 can be designed thinning in a vertical direction (top and/or bottom) towards the locations where the resonance amplitude maximum is desired. The geometries of a membrane 106 can be merged in order to achieve complex geometries 109. The membrane can also be designed to thin in horizontal (sides) direction 111 in order to create predefined flexural wave nodes in order to further amplify the membrane 110 displacement at the distal point. Vertically and horizontally thinning geometries 106, 110 can be combined for further amplification and control of vibrations. These membranes 106, 110 with transducer elements 100, 101 can be sequenced (FIG. 7) to precisely control time difference between activations to achieve sensation of advancing touch. The membranes 110 can also be merged to design complex shapes 112 that are operated with less or only one transducer element 113.

At eigenfrequency antinodes, the vibration of the membrane 102, 104, 105, 106, 107, 110, 112 exhibits sufficient boundary displacements to generate pressure fields. When these pressure fields advance to the air-skin interface, the difference in acoustic impedances results in acoustic radiation force acting towards the skin. This CT-optimal force <5 mN causes tissue deformation at the skin. When shifting between eigenfrequencies, the locations of maximum pressure fields are moved. With appropriate sequencing of eigenfrequencies and adjacent membranes 102, 104, 105, 106, 107, 110, 112, the tissue deformation can achieve CT preferred movement speed in range of 1-10 cm/s with particular preference for speed of 3 cm/s. The size and spatial shift of these pressure fields can be manipulated for high number of stimulation patterns to overcome an overlooked problem of receptive field fatigue to repetitive stimuli. Deconstructive regions are used to define the areas where no stimulation is given, allowing recovery of fatigued receptive fields.

Also, one or more fibers 115 may be coupled to the vibrating membrane 102, 104, 105, 106, 107, 110, 112, 121 so that the membrane vibration is converted to flexural mode 103 in the fibers 115. The flexural wave amplitude at the membrane-fiber interface 114 is dependent on the membrane vibration node's normal direction gradient. In other words, the contact point demonstrates angular force whose magnitude drives flexural mode in a fiber. The fibers 115 may have uniform diameter along the length, or the diameter can decrease (thin) towards the point of the fiber 116. Exponential thinning rates are used to amplify vibration amplitudes in the fibers (15 for increased energy efficiency.

It is important that the fibers 115 are in direct contact or at close proximity with skin's terminal and/or vellus hairs 122. The fibers 115 may be in contact with one or more hair and hair type. The flexural waves 103 in fibers 115 are transferred to body hair through direct contact interface and displacement between the fiber 115 and skin hair 122, or through non-contact interface by generating resonance frequencies in skin's hairs 122. The displacement of skin's hairs 122 causes shear, tensile, and compression forces around the hair follicle, causing tissue deformation is skin's 123 epidermis, basal layer, and dermis capable of C-tactile afferent activation.

FIG. 9 illustrates, by way of an example, an arrangement for a device according to an embodiment. The arrangement comprises two fibers 115 coupled to a transducer element 101 via an impedance matching element 107. Fiber 115 is connected to the transducer element so that a proximal fiber 114 is aligned perpendicular to a surface of the transducer element 101, and narrowing towards its opposing end 116, which is configured to contact with skin hairs. A vibration pattern of the membrane 102 is illustrated as a dashed line 103. The transducer element 101 is configured to oscillate transversely.

FIG. 10 illustrates, by way of an example, an arrangement for a device according to an embodiment. The arrangement comprises two fibers 115 coupled to a transducer element 101 via an impedance matching element 107. Fiber 115 is connected to the transducer element so that a proximal fiber 114 is aligned perpendicular to a surface of the transducer element 101, and narrowing towards its opposing end 116, which is configured to contact with skin hairs. A vibration pattern of the membrane 102 is illustrated as a dashed line 103. The transducer element 101 is configured to oscillate longitudinally.

FIG. 11 illustrates, by way of an example, an arrangement for a device according to an embodiment. In addition to the parts presented in the previous FIG. 9 the arrangement of FIG. 11 comprises an element 117, an element 118, casing 119 and a bridge element 120.

FIG. 12 illustrates, by way of an example, an arrangement for a device according to an embodiment. In addition to the parts presented in the previous FIG. 9 the arrangement of FIG. 12 comprises an element 118 and a casing 119.

As demonstrated by FIGS. 9-12, the fibers 115 may also be directly coupled with a transducer element 100; 101 with impedance matching elements 107. The transducer element 100, 101 providing the displacement can be moving in a direction parallel to its surface 101 or perpendicular 100 to it. Fibers 115 connected to the transducer element can be position so that the proximal fiber 114 is aligned parallel to the transducer surface (FIG. 9; FIG. 11). Alternatively, the proximal fiber 114 can be positioned perpendicular to the surface (FIG. 10; FIG. 12). It is important that the fiber 115 is narrowing towards the site of contact with skin hairs 116 to impedance match the wave propagation 103 converting the force to a displacement, improving the action on the skin hairs 122, and improving sensations by the skin hairs 122. In cases where the fibers 115 bend, the radius r of the bending curve must be in correct proportion with the traversing wave wavelength to enable mode conversion from longitudinally oscillating wave mode at 114 to flexural mode at 116, or to disable mode conversion from flexural mode at 114 to longitudinal mode at 116. The transducer elements 100, 101 may operate in unimodal (one-directional), bimodal (two-directional), or trimodal (three-directional) mode. Employing bi- or trimodal activation enables complex vibration modes in fibers 115. Electronic control unit supplies transducer elements 100, 101 appropriate electrical signal to precisely control transmitted flexural wave acoustic properties in fibers.

FIG. 13 illustrates, by way of an example, an arrangement for a device according to an embodiment. The arrangement comprises a transducer 100, layered membranes 104, 105 and extrusion elements 121. The arrangement is illustrated next to user's skin 123 and skin hairs 122.

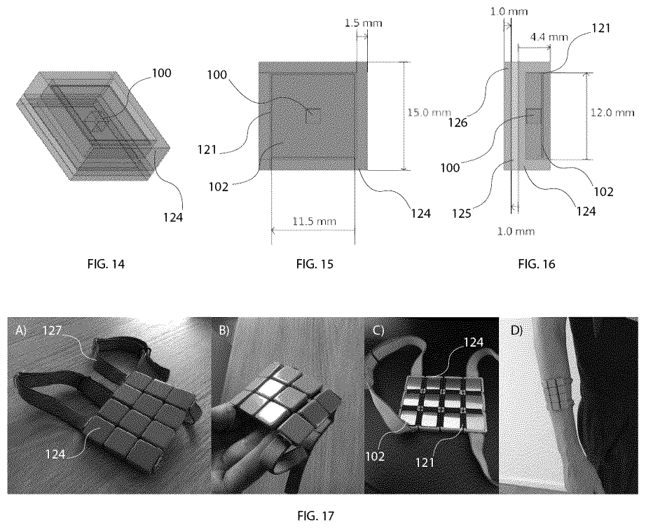

FIG. 14 illustrates, by way of an example, an arrangement for a device according to an embodiment. The arrangement comprises a transducer 100 and a casing 124.

As demonstrated by FIGS. 13 and 14, the longitudinally oscillating transducer elements 100 may be utilized to actuate fibers 115 in transverse mode using an extrusion element 121 as a vibrating membrane or as a waveguide. The extrusion element 121 is coupled to the transducer element 100 only with impedance matching 107 (FIG. 14), or with both impedance matching 107 and bridge elements 120. This enables long but thin (FIG. 15) and stacked but high (FIG. 14) element designs. It is important that the transducer element 100 is coupled to the inner wall of the casing 119 with an element 117 which has much higher acoustic impedance than 100. The bridge element 120 must have the same or close to same acoustic impedance with the transducer 100 and the extrusion element 121. The extrusion element 121 may be free to oscillate or coupled to the inner wall of the casing 119 with an element 118 which has much smaller acoustic impedance than the extrusion element 121.

FIG. 15 illustrates, by way of an example, a top view an arrangement of FIG. 14. The arrangement shows a transducer 100, a membrane 102 and an extrusion element 121.

Demonstrated by FIG. 15, a membrane 102, 104, 105, 106, 107, 110, 112 can be placed in less than 2 mm distance (the average length of a vellus hair 122) from the skin's surface forces vellus hairs 122 to bend and couple physically with the membrane 102, 104, 105, 106, 107, 110, 112. In this condition, the membrane 102, 104, 105, 106, 107, 110, 112 can be vibrated with transducer elements 100, 101, 113 so that the membrane 102, 104, 105, 106, 107, 110, 112 displacement directly generates flexural waves in vellus hairs 122. The membrane 100 can be layered with materials with lower acoustic impedances 104, 105 to amplify vibration, as demonstrated in FIG. 2. The casing 124 has extrusions 121 that maintain the constant distance between the skin's surface 123 and an active element. It is important that the acoustic impedance Z4 of the extrusions 121 is much less than for the most bottom layer 100, 104, or 105, Z1>Z2>Z3>>Z4 to avoid any vibration traversing onto the skin's surface 123 along them and activating wrong afferent nerve fibers.

FIG. 16 illustrates, by way of an example, a side view of an arrangement of FIG. 14. The arrangement shows a transducer 100, a membrane 102, an extrusion element 121, a casing 124 and layers 125, 126.

FIGS. 17A, 17B, 17C and 17D illustrate, by way of an example, a device according to embodiments. The device of FIG. 17A shows multiple casings 124 and straps 127. FIG. 17B shows a top view of the device. FIG. 17C shows a bottom view of the device including membranes 102, extrusion elements 121 and casings 124. FIG. 17D shows the device attached to a user's arm.

The casing 124 comprises the electronic control unit, sensors, transducer elements 100, 101, 113, thin membrane 102, 104, 105, 106, 107, 110, 112, 121 and may also comprise fibers 115. The casing 124 has extruded elements 121 that are used to maintain constant distance between skin's surface 123 and membrane 102, 104, 105, 106, 107, 110, 112, 121, providing appropriate space for generating pressure fields towards the skin, to vibrate vellus hair 122 directly or with vibrating fibers. The extrusions 121, 124 have adhesive properties that are used to maintain constant apparatus positioning on the skin. The coupling with a body location is further improved with tightening straps 127.

The casing (124) is further subdivided into active cells 124 (FIG. 16-18) that comprise active components, including transducer elements 100, 101, 113), thin membrane 102, 104, 105, 106, 107, 110, 112, 121 and/or fibers, 115. Multiple pieces of these active cells 124 can be arranged adjacent to each other to cover large skin area (FIG. 19), enhancing the neuromodulatory effects of C-tactile afferents. The larger area is covered, the stronger the neuromodulatory effect is.

The apparatus has one or more sensors providing electrical signaling to the electronic control unit. The sensors measure distance from the membrane to the skin, and indirect physiological biomarkers, such as heart rate and electrodermal activity, which are used in biofeedback to monitor and enhance apparatus activation. The transducer elements 100, 101, 113 may be activated individually in synchronous or asynchronous patterns based on electrical signaling from the electronic control unit and/or biofeedback.

Currently, there are very limited number of scientifically validated technological solutions that optimally activate C-tactile afferents, CTs. CTs are an essential part of the social touch system and optimally activating them can produce significant alleviation on behavioral disorder symptoms, anxiety, stress, neuropsychiatric, and many other conditions. The existing solutions are typically large, rely on vibration that is not optimal for CT activation, or do not take into consideration the receptive field properties of optimal CT activation, thus limiting their effective use in self-care therapeutic applications. The presented mechanisms of action achieves CT-optimal stimulation through thin geometries, suitable for wearable devices. Wearable CT-activating therapeutic technology enables patients to receive treatment where and whenever needed.

We here describe the solution which is a haptic device for modulation of addiction-, stress- and reward-related brain sites and neurotransmitter systems for therapeutic use in the treatment of alcohol and other substance use disorders, behavioral disorders including behavioral addictions, obesity, anxiety disorders, sleep disorders and stress management.

The device is designed to produce haptic stimulation which results in optimal activation of insular cortex, a brain site that has an important role in addiction-related substance craving. In addition, the device will modulate the endogenous opioid peptide systems, dopamine, oxytocin and cortisol release and the functioning of the hypothalamic-pituitary-adrenal axis, reward circuits and the parasympathetic nervous system.

The device is designed to generate a pleasant haptic effect that simulates gentle stroking touch on the skin. The tactile stimulation created by the device will activate the touch systems including C-tactile afferents (CT) that project to insular cortex which is functionally connected to the brain areas involved in reward and motivation and affects the dopaminergic activity in the ventral tegmental area and the nucleus accumbens.

These above mentioned central and peripheral systems are known to have several positive effects on physiological and behavioral stress responses, as well as other neurobiological mechanisms related to alcohol craving and addictive behavior (FIG. 7). By the modulation of these brain sites and the parasympathetic nervous system this device will be able to build tolerance to stress-induced craving, which is a major contributor to alcohol relapses. In short, it will prevent relapses by reducing impulsive responses to emotional stress.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

The described features, structures, or characteristics may be combined in any suitable manner in one or more embodiments. In the previous description, numerous specific details are provided, such as examples of structures, lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, i.e. a singular form, throughout this document does not exclude a plurality.

1. A haptic device,
comprising an electronic control unit and one or more transducer element,
wherein the one or more transducer element is configured to be activated based on electrical signaling from the electronic control unit, and
wherein the one or more transducer element is configured such that vibrations are obtainable through modifying electrical signaling from the electronic control unit to the one or more transducer element such that the vibrations are configured to generate pressure fields,
further comprising at least one membrane coupled to the one or more transducer element;
wherein a resonance pattern in the at least one membrane is configured to generate spatiotemporally modifiable local displacement regions, which generate acoustic pressure fields respectively.

2. The haptic device according to the claim 1, wherein the one or more transducer element is configured to be vibrated such that one or more eigenfrequency patterns are obtainable in the at least one membrane,
and shifts between the eigenfrequencies are enabled with the same configuration through modifying electrical signaling from the electronic control unit to the one or more transducer elements wherein the shifts between eigenfrequencies cause moving of locations of maximum pressure fields.

3. The haptic device according to the claim 1, wherein the one or more transducer element is configured to operate in a unimodal, a bimodal, or a trimodal vibration mode.

4. The haptic device according to claim 1, wherein the one or more transducer element comprises a piezo element.

5. The haptic device according to claim 1, wherein the one or more transducer element is configured to operate either in transverse or in longitudinal mode.

6. The haptic device according to claim 1, further comprising at least one or more of the following:
wherein the at least one membrane is configured to be vibrated with the one or more transducer element in order to obtain plural different eigenfrequency patterns in the at least one membrane; and
wherein the one or more transducer element is coupled on top of the at least one membrane and/or at sides of the at least one membrane.

7. (canceled)

8. The haptic device according to claim 1, wherein at eigenfrequency antinodes, vibration of the at least one membrane is configured to vibrate such that vibration of the at least one membrane exhibit sufficient boundary displacements to generate pressure fields.

9. The haptic device according to claim 1, further comprising at least one adjacent membrane layered together with the at least one membrane.

10. (canceled)

11. The haptic device according to claim 1, wherein at least two membranes are layered together and the layered at least two membranes are coupled to only one transducer.

12. The haptic device according to claim 9, further comprising the layered membranes wherein the layered membranes are configured to oscillate longitudinally, and wherein the layered membranes having different acoustic impedances.

13. The haptic device according claim 1, further comprising one or more fibers coupled to the one or more transducer element,
wherein the electronic control unit is configured to supply appropriate electrical signal to the one or more transducer element in order to control transmitted flexural mode in the one or more fibers.

14. The haptic device according to claim 13, wherein the one or more fiber has uniform diameter along the length of the fiber, or the diameter decreases towards the point of the fiber.

15. The haptic device according to claim 13, further comprising at least one impedance matching element, wherein the one or more fiber is directly coupled with the one or more transducer element with the impedance matching elements.

16. The haptic device according to claim 1, further comprising a solenoid.

17. The haptic device according to claim 1, further comprising at least two or more casings comprising one or more transducer; with or without one of the following: the at least one membrane, or the one or more fibers;
wherein the at least two or more casings are sequenced in order to control time difference between activations.

18. The haptic device according to claim 1, wherein the haptic device is a wearable device, or the haptic device is comprised in a furniture, or the haptic device a in a brush-like device, wherein vibrated elements arranged against a skin of the user are configured to provide haptic forces.

19. The haptic device according to claim 1, wherein the device further comprises extrusion elements configured to maintain a constant distance between a surface of a skin of a user and the casing.

20. (canceled)

21. The haptic device according to the claim 19, wherein the extrusion element is free to oscillate, and/or
wherein the one or more transducer element is configured to oscillate longitudinally and arranged to actuate the one or more fibers in a transverse mode such that the extrusion element is used as a vibrating membrane or as a waveguide.

22. (canceled)

23. The haptic device according to claim 18, wherein acoustic resonances employed in the at least one membrane are configured to generate pressure fields at a surface of a skin of a user, which is configured to transfer acoustic energy through coupling between the one or more fibers and user skin hair; and
wherein acoustic resonances employed in the at least one membrane are configured to generate pressure fields at air-skin interface, wherein difference in acoustic impedance is configured to result in acoustic radiation force acting towards user skin.

24. The haptic device according to claim 23, wherein the one or more transducer element is configured to generate transverse displacement which generates flexural waves in the one or more fibers, and wherein the flexural waves of the one or more fibers are coupled to skin hairs of the user, which is configured to cause tissue deformation at the hair root and follicle.

25. The haptic device according to claim 17, further comprising plurality of casings in a cellular structure, wherein the activation of the plurality of casings is sequenced in order to provide continuous haptic patterns.

26. The haptic device according to claim 1, further comprising one or more sensors configured to provide electrical signaling to the electronic control unit, wherein the one or more sensors are configured to measure;
a distance; either from the one or more transducer, or from the at least one membrane; to a skin surface of the user, and
biomarkers obtained from the one or more sensors.

27. (canceled)

28. The haptic device according to claim 26, wherein an activity of the obtained biomarkers is used in biofeedback to monitor activation of the haptic device; and
wherein the two or more transducer elements are activated individually in synchronous or asynchronous patterns based on the electrical signaling from the electronic control unit and/or based on the biofeedback.

29. (canceled)
